(19) United States
(12) Patent Application Publication
(10) Pub. No.: US 2016/0015009 A1
(43) Pub. Date: Jan. 21, 2016

(54) UV STERILIZER, TANK SYSTEM USING SAME, AND METHOD OF OPERATING

(71) Applicant: SEALAND HOLDINGS CO., LTD., Kwung Tong (HK)

(72) Inventors: Barry V. PREHODKA, Ridgefield, CT (US); Yat Lung Stephen LAM, Hong Kong (HK); Hong Wa SETO, Kowloon (HK)

(21) Appl. No.: 14/800,397

(22) Filed: Jul. 15, 2015

Related U.S. Application Data
(60) Provisional application No. 62/025,115, filed on Jul. 16, 2014.

(51) Int. Cl. A01K 63/04; A61L 9/20; A01K 63/00

(52) U.S. Cl. CPC A01K 63/045; A01K 63/003; A01K 63/047; A61L 9/20

(57) ABSTRACT

A UV sterilizer. The UV sterilizer has a casing defining an inlet and an outlet therein and a conduit therein between the inlet and the outlet, wherein the casing is capable of conveying a liquid from the inlet through the conduit; a UV electric bulb situated within the casing adjacent the conduit; a power source for supplying electricity to the bulb; and a mechanical fastener for attaching the sterilizer to a pump. The sterilizer is unitary in structure and is substantially free of a pump.

### CROSS-REFERENCE TO A RELATED APPLICATION

The present application claims priority to U.S. Provisional Application Ser. No. 62/025,115, filed Jul. 16, 2014.

### BACKGROUND OF THE DISCLOSURE

1. Field of the Disclosure

The disclosure relates to a UV sterilizer, a tank system using same, and a method of operating a tank system using same.

2. Description of the Related Art

A problem with maintaining fish tanks or aquariums is a buildup of (debris) unwanted microorganisms and algae within the water and surrounding contact surfaces. As fish tanks are typically transparent for viewing purposes and/or aesthetics, buildup is readily apparent and can occur within a relatively short period of time. Microbial diseases such as ich are detrimental to the health of aquatic specimens.

Various methods for (cleaning) removing such undesirable elements from fish tanks are available. One method is to simply change the entire water contents and manually scrub internal surfaces of the tank. This method is effective but unhygienic and messy and has to be performed frequently. This also requires removing livestock from the tank and risking harm to them. Another method is to add antimicrobials, such as antibiotics or metal compounds, to the water periodically. This method can be effective but subject the fish to foreign substances that may affect their health and/or longevity. Another method is to employ filters in conjunction with pumps to filter out debris. This method can be partially effective but requires frequent filter changes. However, filters only mechanically remove debris and can allow live organisms to pass without any harm. Another method is the use of UV (ultraviolet) sterilization in conjunction with a pump as shown in the device in U.S. Pat. No. 7,727,406. Such devices kill live organisms such as algae and pathogens such as ich by exposure of the organisms to light in the ultraviolet range, most effectively at the wavelength of 254 nM. Such devices can be effective, but are typically inserted into the water and exhibit relatively high pressure drop requiring relatively powerful pumps to move the water. Pumps also can fail for many mechanical reasons including clogging with water borne grit.

It would be desirable to have a UV sterilizer that is useful in containing the buildup of algae and undesirable microbial buildup in fish tanks. It would further be desirable to have a UV sterilizer that maintains a clean and hygienic environment for fish living in the tank. It would further be desirable to have a system that effectively employed a UV sterilizer. It would further be desirable to have a method of operating a tank system using a UV sterilizer without the need for a motor or pump system.

### SUMMARY OF THE DISCLOSURE

According to the present disclosure, there is provided a UV sterilizer. The UV sterilizer has (a) a casing defining an inlet and an outlet therein and a conduit therein between the inlet and the outlet, wherein the casing is capable of conveying a liquid from the inlet through the conduit; (b) a UV electric bulb situated within the casing adjacent the conduit; a power source for supplying electricity to the bulb; and (c) a mechanical fastener for attaching the sterilizer to an existing pump. The sterilizer is unitary in structure and is substantially free of a pump.

According to the present disclosure, there is provided a fish tank system. The system has a tank containing water and livestock and a pump capable of recirculating the water of the tank and situated above a water line of the water. The UV sterilizer is situated above the water line and below and adjacent the pump. The pump is capable of conveying water via gravity to the UV stabilizer. The UV stabilizer is capable of exposing the water to UV radiation therein.

According to the present disclosure, there is provided a method for operating a fish tank system. The method has the steps of (a) filling a tank with water and livestock; (b) suctioning water from the tank with a pump situated above a water line of the water; and (c) conveying it via gravity to and through a UV sterilizer situated above the water line and below and separate from the pump.

### DESCRIPTION OF THE FIGURES

FIG. 1 depicts an oblique view of a UV sterilizer according to the present disclosure.

FIG. 2 is a rear view of the UV sterilizer of claim 1.

FIG. 3 is a cross-sectional view of the UV sterilizer of claim 1 taken along line 2-2.

FIG. 4 is an end view of the UV sterilizer of claim 1.

FIG. 5 is a cross-sectional view of the UV sterilizer of claim 1 taken along line 4-4.

FIG. 6 is a top view of the UV sterilizer of claim 1.

FIG. 7 is a bottom view of the UV sterilizer of claim 1.

FIG. 8 is bottom view of a UV sterilizer together with electric cord, adapter, and electric plug.

FIG. 9 depicts a tank system according to the present disclosure in operation.

### DETAILED DESCRIPTION OF THE DISCLOSURE

The UV sterilizer of the present disclosure exposes tank water to ultraviolet radiation (light) and is useful in containing or limiting the buildup of algae and excessive antimicrobial buildup in fish tanks. The containment of buildup reduces the frequency with which the tank water has to be changed and the interior surfaces of the tank manually cleaned. The UV sterilizer helps to maintain a clean and hygienic environment for fish living in the tank.

The UV sterilizer is unitary and integral in structure and constructed separately and apart from the pump and mechanical filter. The UV sterilizer is attached, connected, or affixed to the pump by conventional mechanical fasteners. Useful fasteners include clips, Velcro, pins, magnets, and suction cups. Clips are preferred.

The UV sterilizer is situated or positioned within the tank system at the exit of the filter and pump system. Such filter systems use a small motor that pumps water above the water level of the fish tank, pushes the water through mechanical filters, such as those of foam, activated charcoal and small rocks. Water is then returned to the tank from the filter from above the tank water level via gravity. This gravity returned water can be utilized to convey the recirculated water to and through the UV sterilizer. The use of gravity flow water enables a steady slow flow of water over the UV bulb. The UV sterilizer is preferably structured such that the pressure drop induced upon water flowing therethrough is small. Small water passage clearances between the bulb, bulb housing, and baffles ensure a slow steady flow of water against the glass surface of the UV bulb. The clip attachment means allow the user to selectively attach the UV unit to the output of the filter to reduce tank contaminants as needed.

The pump and UV sterilizer are preferably sized so that the water in the tank is treated at least every 2 to 3 days, although treatment regimes of longer or short times are possible.

The bulb in the UV sterilizer that emanates ultraviolet radiation is preferably sized and is of sufficient intensity to kill or diminish/reduce the active population level of microbial organisms within the tank water as it traverses the UV sterilizer. Size and/or intensity of the bulb/lamp will vary depending on the throughput requirements of the tank system. For aquariums of conventional volume ranges, bulbs of about 2 to about 50 rated wattage will be typical. Preferably, the bulb is situated or positioned in the UV sterilizer such that traversing water directly contacts the bulb. However, it is also possible to position a layer(s) of UV-transmissible materials between the bulb and the traversing water so that the bulb does not get wet. As UV light is detrimental to the human eye, it is also important to construct the assembly to prevent the user from being able to see the UV light rays.

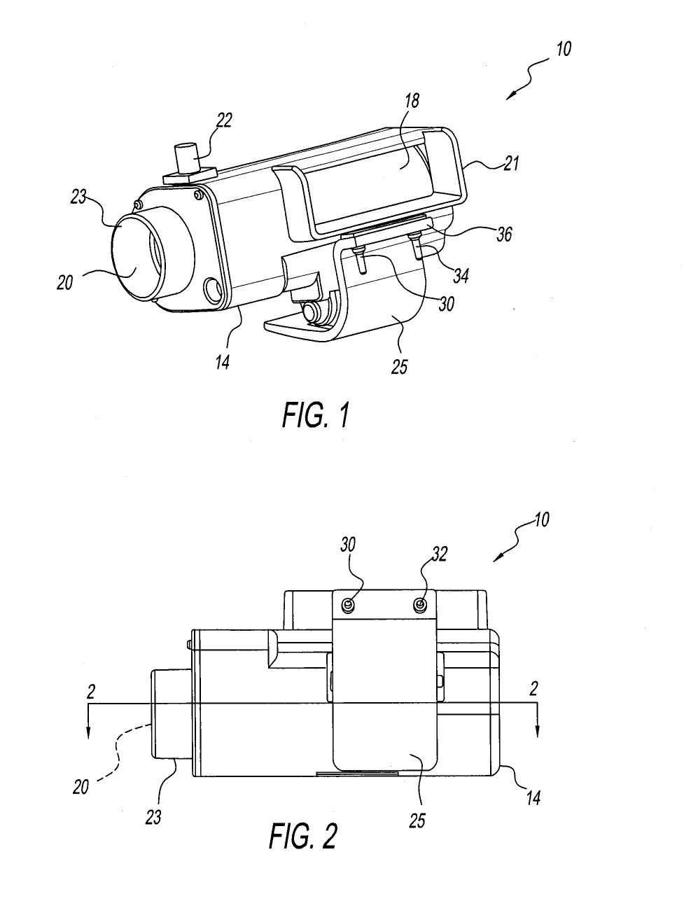

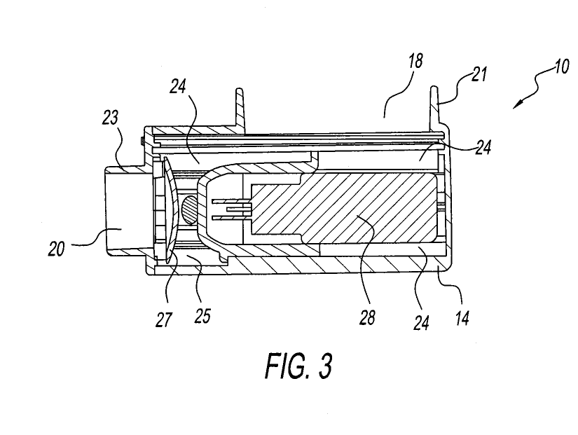

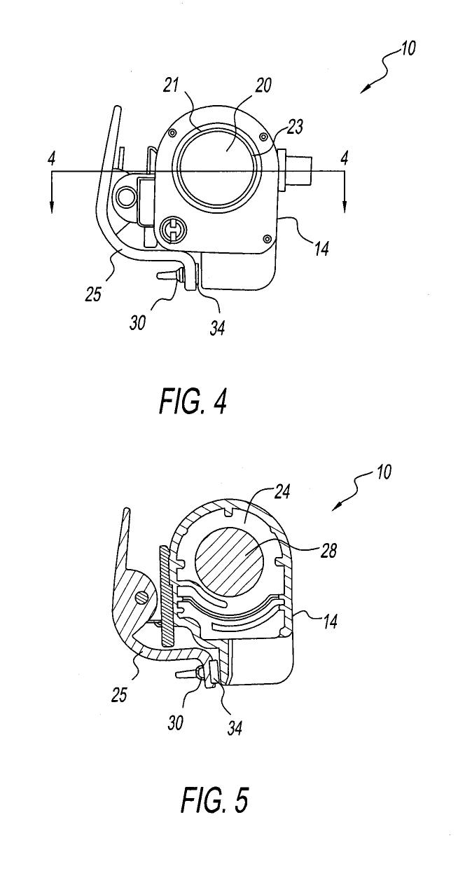

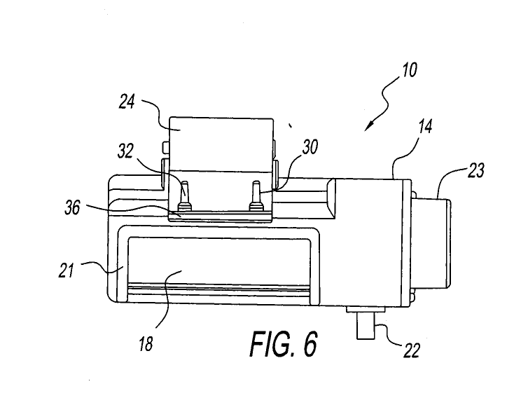

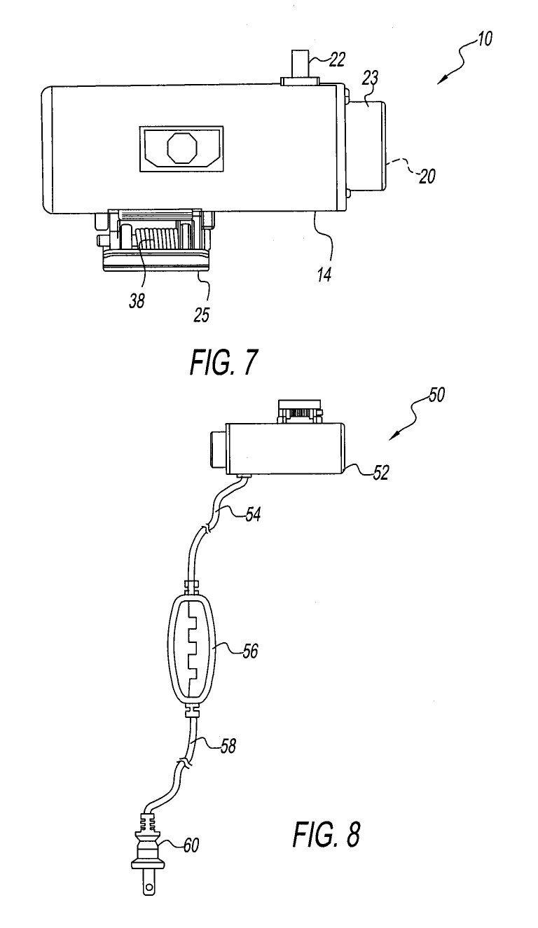

A UV sterilizer of the present disclosure is shown and described in FIGS. 1 to 7 and is generally referenced by the numeral 10. UV sterilizer 10 has a casing 14 defining an inlet 18 and an outlet 20 therein and a conduit 24 therethrough between inlet 18 and outlet 20. Casing 14 is capable of receiving conveying a liquid (tank water) from inlet 18 through conduit 24 to outlet 20. Inlet 18 is bounded by a flange 21 at the exterior of casing 14 that creates a larger inlet for receipt of liquid. Outlet 20 is bounded by cylindrical portion 23 at the exterior of casing 14. Outlet 20 is bounded by circular curved portion 27 around the periphery of which liquid flows from conduit 24 into outlet 20. A UV electric bulb 28 is situated within casing 12 adjacent conduit 24. A power source (not shown) supplies electricity to bulb 28 through a receptacle 22. A clip 25 is used to attach sterilizer 10 to a pump (not shown). Sterilizer 10 is unitary in structure and is substantially free of and not integral with a pump (not shown). Clip 25 rotates resistively around a coil spring mechanism 38. Clip 25 can be utilized by pressing inward a proximal portion 38 of clip 25 and releasing upon engagement of clip 25 with a pump (not shown) at a distal portion 36 of clip 25. A cushioning pad 34 is attached to clip 25 by a pair of pins 30 and 32 at a distal portion 36 of clip 25. Clip 25 engages a pump (not shown) at the juncture of distal portion 36 and casing 14.

A UV sterilizer system of the present disclosure is shown and described in FIG. 8 and is generally referenced by the numeral 50. UV system 50 has a UV sterilizer 52, electric cords 54 and 58, an adapter 56, and an electric plug 60. Adapter 56 steps down the voltage from a conventional household electrical outlet (not shown) into which electric plug 60 is plugged.

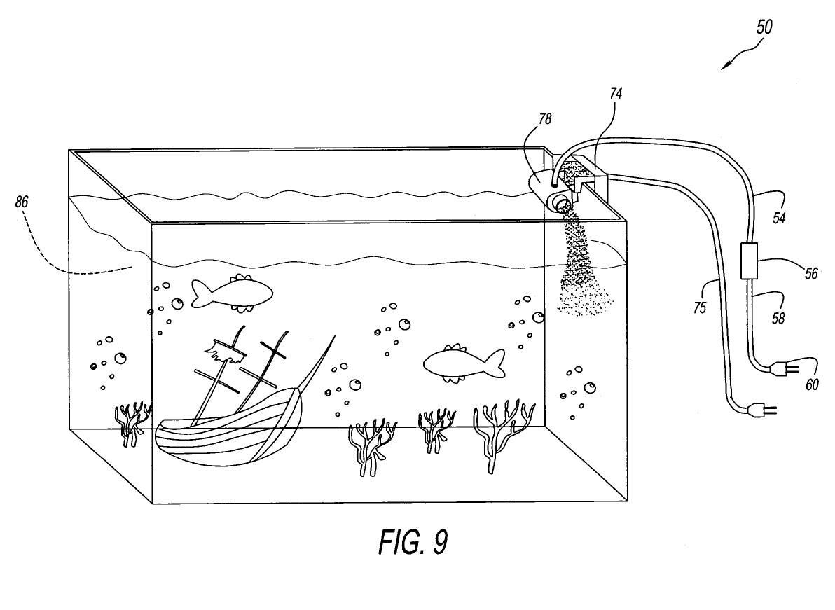

A tank system of the present disclosure is shown and described in FIG. 9 and is generally referenced by the numeral 70. Tank system 70 has a pump and filter system 74, a UV sterilizer 78, and a tank 82 with water 86 therein. Pump 74 is connected to an electric cord 75 and electric plug 77, which is plugged into a household electrical outlet (not shown). UV sterilizer 78 has electric cords 54 and 58, an adapter 56, and an electric plug 60. Pump 74 is separate from and is situated above UV sterilizer 78 and conveys water to UV sterilizer 78 via gravity. UV sterilizer 78 is clipped onto pump 74. Gravity is sufficient to enable water to pass through UV sterilizer 78 given the relatively low pressure drop induced on water 86 traversing UV sterilizer 78.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

What is claimed is:

1. A UV sterilizer, comprising:
a casing defining an inlet and an outlet therein and a conduit therein between the inlet and the outlet, wherein the casing is capable of conveying a liquid from the inlet through the conduit;
a UV electric bulb situated within the casing adjacent the conduit;
a power source for supplying electricity to the bulb; and
a mechanical fastener for attaching the UV sterilizer to a pump,
wherein the UV sterilizer is unitary in structure and is substantially free of a pump.

2. The sterilizer of claim 1, wherein the bulb is situated within the conduit.

3. The sterilizer of claim 1, wherein the bulb is separated from the conduit by a layer of UV transmittable material.

4. The sterilizer of claim 1, wherein the mechanical fastener is a clip.

5. The sterilizer of claim 1, wherein the mechanical fastener is for attaching the UV sterilizer to a pump with a filter system, wherein the UV sterilizer is further substantially free of a filter system.

6. A fish tank system, comprising:
a tank containing water and live fish and
a pump capable of recirculating the water in the tank and situated above a water line of the water or adjacent to and outside the tank,
wherein the UV sterilizer is situated above the water line and below and adjacent the pump, wherein the pump is capable of conveying water via gravity to the UV stabilizer, wherein the UV stabilizer is capable of exposing the water to UV radiation therein.

7. A method for operating a fish tank system, comprising:
filling a tank with water and live fish;
suctioning water from the tank with a pump situated above a water line of the water and conveying it via gravity to and through a UV sterilizer situated above the water line and below and separate from the pump.

8. The method of claim 7, wherein the UV sterilizer is mechanically fastened to the pump.

9. The method of claim 8, wherein the UV sterilizer is mechanically fastened to the pump with a clip.

10. The method of claim 8, wherein the pump and the UV sterilizer are situated within the tank.

11. The method of claim 8, wherein the pump is situated adjacent to and outside the tank.
